(19) United States
(12) Patent Application Publication
(10) Pub. No.: US 2005/0283202 A1
(43) Pub. Date: Dec. 22, 2005

(54) Neuromodulation system

(76) Inventor: Barry N. Gellman, N. Easton, MA (US)

Correspondence Address: FAY KAPLUN & MARCIN, LLP, 15O BROADWAY, SUITE 702, NEW YORK, NY, 10038, US

(21) Appl. No.: 10/873,327

(22) Filed: Jun. 22, 2004

(51) Int. Cl. A61N 1/18

(52) U.S. Cl. 607/48

(57) ABSTRACT

An electrical stimulation system comprises an implant adapted for insertion into a body in contact with target tissue, the implant including a transdermal receiver and an external driver including a source of stimulation current and a transdermal transmitter transmitted the stimulation current to the transdermal receiver. A method of electrically stimulating target tissue comprises surgically implanting an implant with an electrode of the implant adjacent to the target tissue, the implant including a receiver, placing an external driver including a transmitter on a target portion of skin in proximity to the receiver, providing power to the implant portion transdermally from the external driver via the transmitter and the receiver and applying a stimulation current to the target tissue by supplying power received from the external driver to the electrode.

### BACKGROUND INFORMATION

Many debilitating medical conditions may be greatly improved by stimulating targeted nerves and muscles within a patient's body. This stimulation may take the form of an electric current applied directly to a nerve or a bundle of nerves to disrupt the signals carried by those nerves, or to affect the muscles controlled by those nerves. One common example of neural stimulation is the cardiac pacemaker. These pacemakers are implanted in the patients' bodies, near the heart muscle, to provide electrical stimulation of the cardiac muscle so that it contracts at a regulated rate. Once implanted, such a neuromodulation device may operate independently for as long as its power supply is active, or may be selectively activated either by the patient or by other means.

In conventional applications, the ability of a neuromodulation system to operate over extended periods of time has been limited by the endurance of its power supply. Often, the power supply consists of one or more batteries attached to components of the neurostimulation system which are implanted within the body of the patient. Although this approach works well for devices that are used for short periods of time, it can be problematic with devices required to operate beyond the life span of available batteries. In these cases, additional surgery may be necessary to replace the worn batteries. Alternatively, an external power supply carried by the patient may be connected to the implanted device by electrical wires or other types of connections. This simplifies the provision of long term power to the implanted components. However, the patient may be subject to infections and other negative reactions at the location(s) where connections penetrate the skin.

Many other medical conditions may benefit from a direct application of electrical stimulation to muscles, nerves or to the spine. In addition, electrical stimulation of nerves in a patient may be used to disrupt signals carried by those nerves. This may be done, for example, to control pain felt by a patient and to reduce discomfort due to surgery, illness or injury.

### SUMMARY OF THE INVENTION

In one aspect, the present invention is directed to an electrical stimulation system comprising an implant adapted for insertion into a body in contact with target tissue, the implant including a transdermal receiver and an external driver including a source of stimulation current and a transdermal transmitter transmitted the stimulation current to the transdermal receiver.

The present invention is further directed to a method of electrically stimulating target tissue, comprising surgically implanting an implant with an electrode of the implant adjacent to the target tissue, the implant including a receiver, placing an external driver including a transmitter on a target portion of skin in proximity to the receiver, providing power to the implant portion transdermally from the external driver via the transmitter and the receiver and applying a stimulation current to the target tissue by supplying power received from the external driver to the electrode.

### BRIEF DESCRIPTION OF DRAWINGS

FIG. 1 shows a diagram of a neuromodulation system according to an embodiment of the present invention;

FIG. 2 shows a diagram of a neuromodulation system according to a second embodiment of the present invention;

FIG. 3 shows a diagram of the architecture of an implant and of an external component according to an embodiment of the present invention.

FIG. 4 shows a diagram of a neuromodulation system according to a third embodiment of the present invention;

FIG. 5 shows a diagram of a neuromodulation system according to an embodiment of the present invention, including a sensor feedback;

FIG. 6 shows a diagram of a neuromodulation system according to an embodiment of the present invention, including radio signal telemetry;

FIG. 7 shows a diagram of a neuromodulation system according to an embodiment of the present invention, including a sensor feedback and radio signal telemetry;

FIG. 8 shows a diagram of a neuromodulation system according to an embodiment of the present invention, including neuro feedback and signal telemetry;

FIG. 9 shows a diagram of a neuromodulation system according to an embodiment of the present invention, including neurofeedback, signal telemetry and a bio sensor;

FIG. 10 shows a schematic drawing of an embodiment of an implanted component of a neuromodulation device according to the present invention;

FIG. 11 shows a schematic drawing of a distal portion of an implanted component according to another embodiment of the present invention; and

FIG. 12 shows a schematic drawing of a different embodiment of an implanted component according to the present invention.

### DETAILED DESCRIPTION

The present invention may be further understood with reference to the following description and the appended drawings, wherein like elements are referred to with the same reference numerals. The present invention relates to medical devices used to block nerve signals and to provide nerve stimulation. In particular, the present invention relates to nerve stimulation devices that comprise a first portion implanted into a patient, and a second portion external to the patient but connected to the implanted portion via a transdermal coupling.

As described above, many medical conditions may be alleviated by directly intervening with the nerves leading to and from affected organs. In particular, ailments involving muscle spasms, muscle weakness and irregular or improper activation of muscles may benefit from the present system. One common example of a neuroactivation system is a cardiac pacemaker. Pacemakers are generally implanted near a patient's cardiac muscle to artificially maintain a desired cardiac rhythm. An implanted battery pack module is placed within the patient coupled to an electrode which ends in the cardiac muscle. Timed discharges of electric current are released by the device to cause the hearth muscle to contract at the desired rate.

In addition to controlling cardiac rhythm, neurostimulation devices may be used to treat incontinence, sexual dysfunction and to help with pain management, among other things. As described above, such devices typically include a battery pack to provide power and an electronic module determining when to administer current to selected nerve(s). Often, these devices tie into major nerve branches which extend from the patient's spine, and require complex operations to be successfully implanted. In conventional systems, the power supply is provided together with the nerve stimulation components, and is implanted within the patient. This technique involves some obvious drawbacks when the implant is necessary for a time period greater than the life span of the power supply. Conventional solutions involve surgically accessing the implant to regenerate or replace the power supply (e.g., by replacing one or more spent batteries). Alternatively, the power supply may be kept external to the body where it is more easily accessible, at the expense of an increased possibility of infection and/or irritation at the point(s) of entry of the power conductors to the body.

The present invention is directed to a system including an implanted component, an external component and a transdermal coupling. The implanted component is placed within the patient's body either surgically or percutaneously and includes a nerve couple placed in close proximity to a targeted nerve or bundle of nerves. The external component comprises a driver portion affixed to the skin, but which does not penetrate it to reduce the possibility of infection and/or irritation associated with conventional systems using conductors which penetrate the skin. The external component and the implanted component are connected to one another via a transdermal coupling which may include, for example, a radio frequency coupling, an induction field coupling, an electric field coupling, a microwave transmitter or a device employing any other method of wirelessly conveying power across a patient's skin.

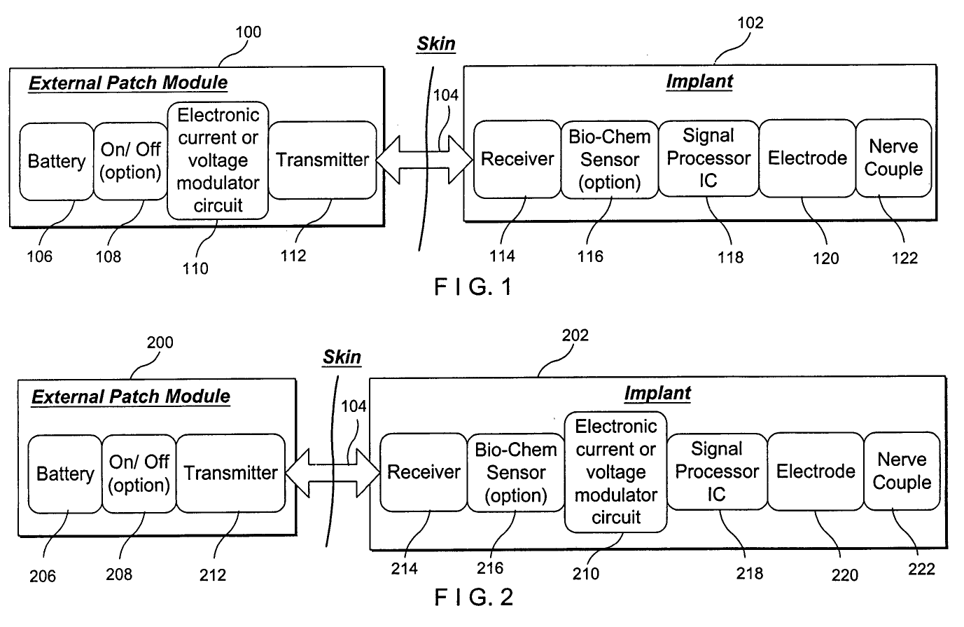

According to embodiments of the present invention, a simplified system for carrying out neuromodulation and neurostimulation is described in FIG. 1. As shown, the system includes an external driver component 100 which may be formed as a patch attached to the skin via an adhesive or kept in place by a bandage. Preferably, the external driver component 100 is thin to minimize interference with the patient's activities. Alternatively, a larger electronic controller may be utilized by providing a means to support it in proximity of the implanted component 102. For example, a PDA or other electronic device with transmitting capabilities may be attached to a belt to place it in proximity to the implanted receiver. The implanted component 102 is surgically placed within the body in proximity to a nerve or nerves to be effected. The implanted component 102 may be made smaller than conventional implants as it does not include a power supply and the electronic components thereof are well suited for miniaturization. As would be understood by those of skill in the art, the transdermal coupling 104 which connects the implanted component 102 to the external component 100 may employ any of the various known techniques for transferring electrical energy through a patient's skin without breaching or injuring the skin. For example, the transdermal coupling 104 may include a radio wave transmitter or a magnetic induction system to transfer electrical energy from the external component 100 to the implanted component 102.

In one embodiment, the external component 100 comprises a battery 106 which provides the power for the entire system. The battery 106 may be a conventional long duration battery, or a rechargeable battery. As would be understood by those skilled in the art, the actual number and type of batteries used may vary depending on system requirements and may preferably be selected to minimize the weight and/or bulk of the external component 100. The external component 100 may also include an on/off switch 108 permitting the patient to control operation of the system and a modulator circuit 110 controlling either or both of an amount of electrical energy to be delivered to the nerve endings by the implanted component 102 and a duty cycle or timing of the delivered current/voltage. This allows the system to deliver selected amounts of amount of energy at selected times. The modulator circuit 110 may also use controllable parameters which are set by the user prior to the procedure to affect the energy output of the implanted components. The transdermal coupling 104 includes a transmitter 112 included in the external component 100 and a receiver 114 included in the implanted component 102 to convey power wirelessly from the battery to the implanted component 102 through the patient's skin.

The external component 100 may be designed as a disposable unit, to be used by a single patient and then discarded. Depending on the amount of power required and the intended duration of treatment, among other parameters, the external component 100 may be fitted with disposable, replaceable batteries, rechargeable batteries, or the external components may be completely disposable once the batteries have been exhausted. Since the external component 100 is not placed within the body, it does not have to be maintained sterile and is not subjected to the harsh environment within the body. Accordingly, the external component 100 can be made relatively inexpensive and can be provided as a disposable component.

As described above, the implanted component 102 comprises a receiver 114 adapted to receive electrical energy sent from the external component 100 via the transmitter 112. A biochemical sensor 116 may be optionally included in the implanted component 102 to monitor the patient before or after activation of the implanted component 102, as will be described more fully below. A signal processor 118 may also be included in the implanted component 102 to refine the modulated current or voltage received from the external component 100 and to channel that energy to a nerve couple 122 via an electrode 120. As would be understood by those skilled in the art, the closer the implanted component 102 is placed to the target nerve or nerve bundle, the shorter the electrode 120 may be made. In other embodiments, the electrode 120 may extend a considerable distance to connect the receiver 114 and the signal processor 118 to a remote nerve couple 122. The nerve couple 122 provides an interface with the patient's biological tissue, and considerable surgical expertise may be required to effectively attach the nerve couple 122 to the target nerve(s).

Additional sensors may be utilized alone or with the biochemical sensor 116, such as pressure and voltage sensors and any other sensor that may provide useful data. In addition, feedback loop control may be employed to adjust the energy delivered based on the sensed parameters. Multiple electrodes, signal processor modules and other components of the device may be placed on, or may be transdermally connected to one or more patches. The implanted components and the external components may be designed to work with a wide variety of corresponding devices, to easily provide the desired level of control and monitoring of the medical procedure.

FIG. 2 shows another exemplary embodiment of a neuromodulation system according to the invention. The principal difference shown in this embodiment is that the modulator circuit 110 has been removed from the external component 100 and a modulator circuit 210 has been added to the implanted component 202. Modulator 210, like its counterpart described with reference to FIG. 1, controls the timing, strength and modulation of the electrical energy being applied to the nerve(s). As will be described below, these parameters may vary depending on the purpose of the neuromodulation system and the anatomical structure to be treated. For example, the energy modulation used for pain suppression is different than the modulation most effective to treat incontinence. Placing the electronic modulator in the external component 100 allows the size of the implanted component 102 to be reduced.

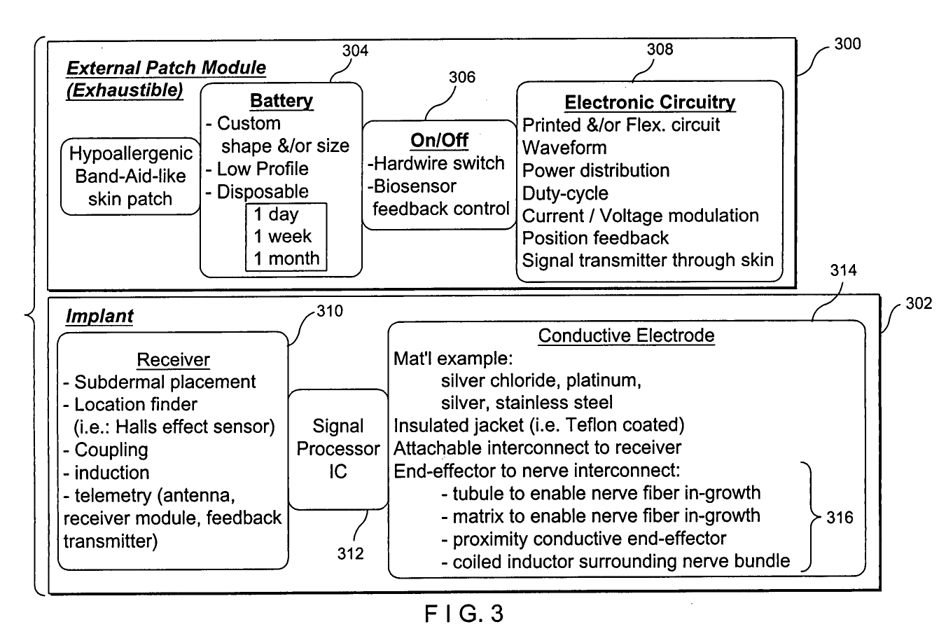

A further exemplary external component 300 and an exemplary implanted component 302 are shown in FIG. 3. The external component 300 may be in the form of a thin skin patch, similar in size and shape to a band aid so that the external component conforms to the curvatures of the portion of the patient's body to which it is attached. For example, the external component 300 may be formed of a hypoallergenic material that can remain on the skin for extended periods of time. A battery 304 contained therein may be custom shaped to fit in the low profile patch. For example, a film battery may be suitable, due to its ability to bend and its sheet-like shape. The battery 304 may be either disposable or rechargeable, with a charge life preferably lasting on the order of days, weeks or months. As would be understood by those skilled in the art and as described above, an on/off switch may be optionally provided and may be patient activated, may follow a preset schedule, or may be coupled to a biosensor in the patient's body to activate the implanted component 302 in response to a predetermined biological event, etc.

The electronic circuitry 308 may be formed on a printed circuit board or on a flexible membrane to better fit in a flexible patch. The circuit 308 may determine the waveform of the current/voltage delivered to the nerves, the power distribution and duty cycle of the device, as well as the type of modulation applied to the current or voltage being administered, depending on the desired treatment method.

The implanted component 302 may also send signals to the electronic circuit 308 relating to, for example, a position feedback. The entire implanted component 302 may be placed subdermally, and may include a device to facilitate positioning the implanted component 302 from outside the body. For example a Hall's effect sensor may be used to provide to the external component 300 (or any other suitable receiver) an indication of the current position of the implanted component 302. As described above in regard to the previous embodiments, the exemplary implanted component 302 includes a receiver 310 adapted to form a transdermal coupling with the transmitter 308. An induction component may be used in the receiver 310 to receive electrical energy from the transmitter in the electronic circuitry 308. In addition, an antenna, receiver module and feedback transmitter may be included in the implanted component 302 to provide additional telemetry options. A signal processor circuit 312 may be used to transform signals received by the receiver 310 into electric charges to be administered to the selected tissue via a conductive electrode 314. The signal processor circuit 312 may comprise an integrated circuit programmed to generate desired electrical impulses based on input received from outside the body or from sensors within the body.

A conductive electrode 314 may be coupled to the implanted component 302 to deliver modulated electrical stimuli to the patient's nerve or nerve bundle. For example, the electrode 314 may be made of silver chloride, platinum, silver, stainless steel or other material that is biocompatible and is a good electrical conductor. An insulated jacket may be placed around the electrode 314 to prevent delivery of the electrical stimuli to tissues that are not targeted. For example, the insulated jacket may be formed of Teflon, or any other biocompatible polymeric or ceramic-based insulator. The electrode 314 may have a length determined by the distance between the receiver 310 and a nerve couple 316 to be located at the distal end of the electrode 314. For many applications, the receiver 310 preferably is located just below the skin so that implantation is simpler and signals from outside the body can be received more efficiently. The nerve couple, on the other hand, is preferably positioned deeper within the body near the target region of tissue.

Various designs of the nerve couple 316 may be used within the scope of the embodiments of the present invention. In one embodiment, the nerve couple 316 may include a proximity conductor which is placed near the target nerve, to impart a desired stimulating electrical current and voltage to the targeted nerve. Those skilled in the art will understand that either or both of the voltage and the current may be varied to achieve a desired stimulation of the targeted tissue. In a different embodiment, an inductor such as a coil may be positioned around a nerve or a nerve bundle to more effectively impart the energy thereto. Configurations of the nerve couple 316 may be used to promote the growth of nerves in or around the electrode. For example, a tubule or a matrix to promote in-growth of nerve fibers may be used at the distal tip of the electrode 314. In yet another embodiment of the present invention, the electrode 314 may interface directly with muscle fibers of a target muscle, to affect some desired control of the muscle. Those skilled in the art will understand that, where the desired end result is to impact a muscle or muscle group, treatment may involve targeting muscle fibers directly by applying the electrical energy to the muscle tissue itself or indirectly by stimulating nerves attached to those muscles.

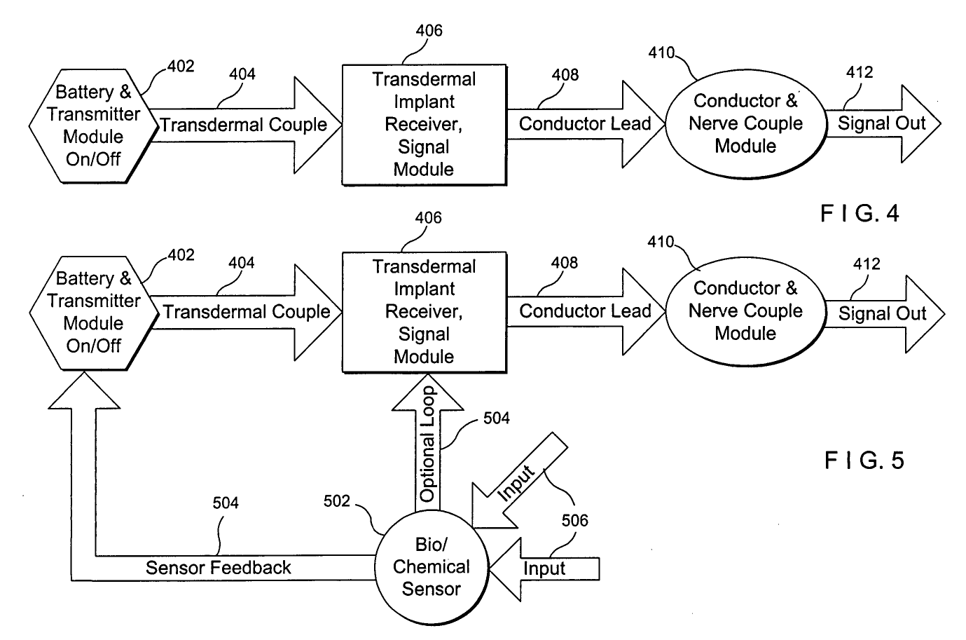

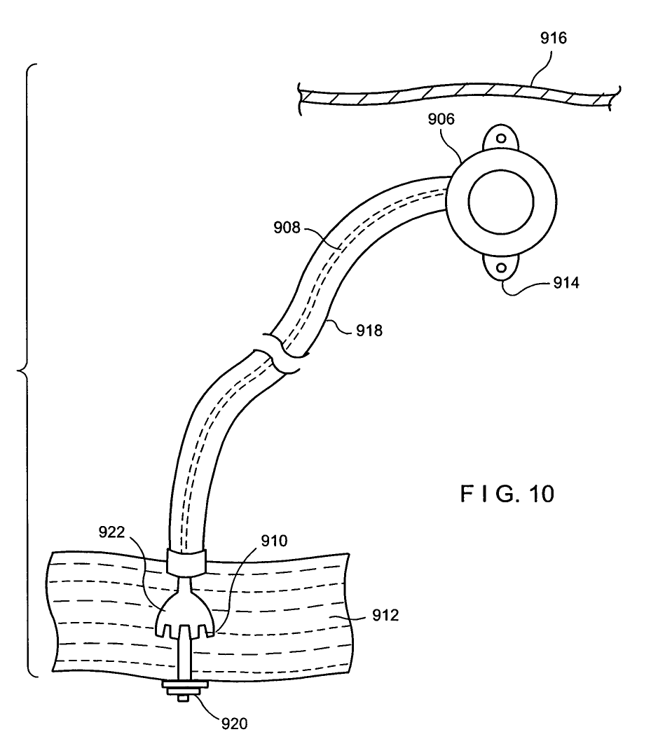

FIG. 4 shows a block diagram of an embodiment of a transdermal neuromodulation system according to the invention. Here, an external electronics module 402 containing, for example, a battery, a transmitter and an on/off switch is connected to an implanted electronics module 406 via a transdermal conduction couple 404. As described above, the couple 404 transmits electrical power to the implanted components without piercing the patient's skin. As described above, implanted electronics module 406 may be a transdermal implant that comprises a receiver and a signal processor module. In addition, a conductor lead 408 may be used to connect the implanted electronics module 406 to a nerve couple 410, from which a signal 412 is used to stimulate the target nerve. FIG. 10 shows an exemplary diagram of implanted elements of a transdermal neuromodulation device, where an implanted electronics module 906 is placed near the patient's skin, and is connected to a nerve couple 910 through a conductor 908.

A feedback control may be used according to other embodiments of the invention, to initiate or to modulate an electrical stimulation signal to be applied to a nerve. As shown in FIG. 5, the feedback may be provided to either the external electronics module 402, the implanted electronics module 406 or to both. A biological or chemical sensor 502 may be used to receive input 506 from surrounding tissues and organs and may use a feedback loop 504 to report to the implanted electronics module 406 and to the external electronics module 402. Specific applications of this feedback loop will be described below.

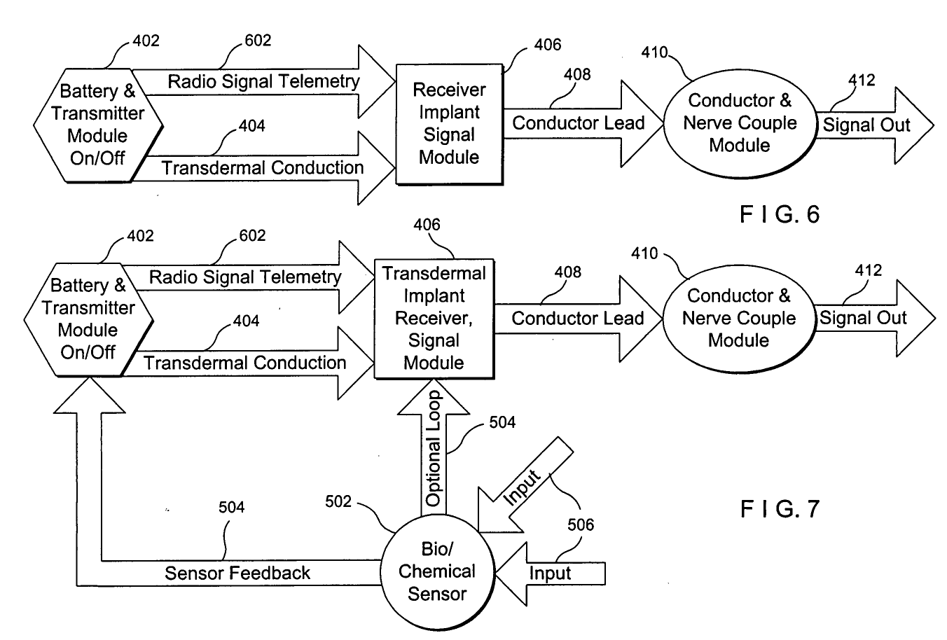

As shown in FIG. 6, control signals may be sent transdermally from the external electronics module 402 to the implanted electronics module 406. For example, a radio signal telemetry 602 may be sent, in addition to power delivery via the transdermal conduction couple 404. In this manner, the implanted components are not only powered via the external electronics module 402, but also receive command signals controlling the operation of the implanted electronics module 406, such as on/off, modulation and intensity commands. The radio signal telemetry 602 is preferably broadcast in the form of radio waves. However other methods of wireless transmission may be used. A visual or audible warning system may be utilized to indicate when the position of the internal and external components is such that the transdermal coupling is possible.

Another embodiment of the present invention, shown in FIG. 7, combines several elements described above. In this example, a biological or chemical sensor 502 is optionally connected to one or both of the implanted electronics module 406 and the external electronics module 402. Both electrical power and telemetry signals are sent from the external electronics module 402 to the implanted electronics module 406. A more sophisticated control of the electrical stimulation delivered to nerves is possible with this configuration, because not only is biosensor feedback provided to one or both of the implanted electronics module 406 and the external electronics module 402, but external commands controlling the electrical stimulation to be delivered may also be sent to the implanted electronics module 406 and may be adapted in response to different situations and optimized in response to changes sensed within the body.

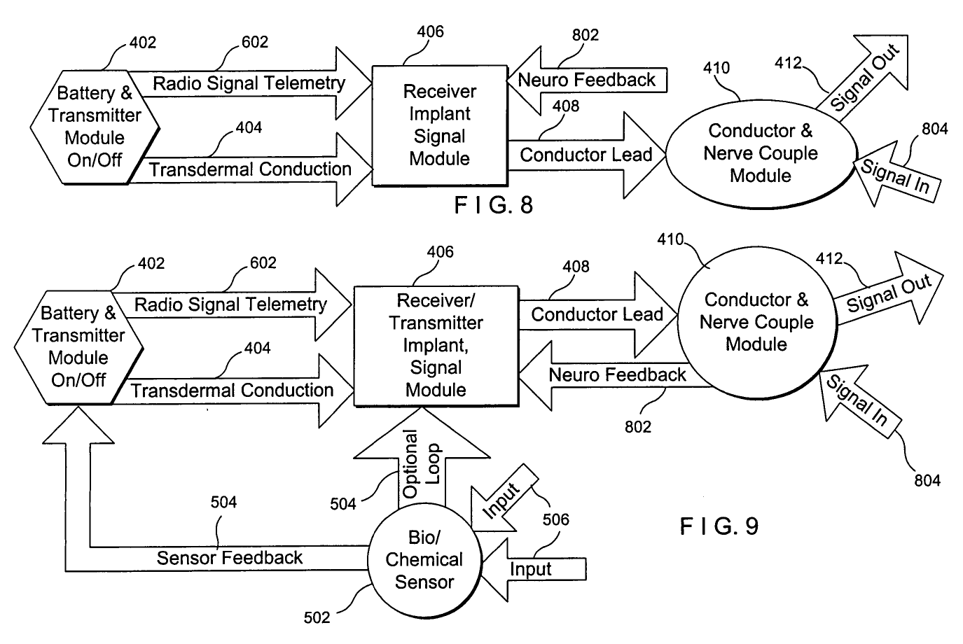

The implanted electronics module 406 may be designed to also receive feedback input directly from the nerve being stimulated in addition to, or in place of, external signals transmitted from the outside via the radio signal telemetry 602, or from feedback provided by the biochemical sensor 502. FIG. 8 shows an example of such an arrangement, where the implanted electronics module 406 receives input from the radio signal telemetry 602 and also from a neuro feedback signal 802. The signal 804 is generated within the nerves of the patient and is received by nerve couple 410, which passes it to the implanted electronics module 406. Since nerve signals are essentially electrical in nature, an electrically conductive nerve couple 410 may also be used to monitor those nerve signals. A feedback loop can thus be set up to monitor the patient's condition. As in other exemplary embodiments of the invention, the power to operate the implanted electronics module 406 is received from the external electronics module 402 through the transdermal couple 404.

FIG. 9 shows an embodiment of the present invention which takes advantage of all the signal input options described so far. The external electronics module 402 contains the power supply and, through the transdermal conduction couple 404, provides that power to the implanted electronics module 406. The radio signal telemetry 602 is also used to control some of the operations of the implanted electronics module 406. A biological and/or chemical sensor 502 is provided within the body to determine certain parameters of the patient's condition and to provide feedback to either or both of the external electronics module 402 and the internal electronics module 406 through the feedback loops 504. In addition, some control of the implanted electronics module 406 is also based on the neuro feedback 802 as nerve couple 410 receives nerve impulses 804 and passes them on to the implanted electronics module 406. This type of system allows the neuromodulation control to be optimized in response to chemical changes within the patient's body and to changes in the signals carried by the patient's nerves.

Embodiments of the system according to the present invention may be used to treat various ailments. For example, damage to the pudendal nerve(s) due to non nerve sparing radical prostatectomy may be alleviated. This nerve damage may result in an inability of male patients to obtain sexual arousal. An electrode such as the nerve couple 410 described above may be placed at time of surgery near the severed pudendal nerve(s), and the appropriate electrical modulation may be applied to stimulate the nerve. A nerve channel may be used as part of the nerve couple, to stimulate nerve fiber growth into a conductive electrode connected to a receiver of an implanted electronics module. A transdermal patch containing the external electronics module, a battery and an on/off switch may be used by the patient. The transdermal patch is placed externally in proximity of the implanted receiver, to provide power and control signals to the implanted components. The patch may have battery life of about a week, and may be recharged or discarded thereafter.

Erectile dysfunctions not caused by vascular deficiencies may also be treated using the system according to the present invention. Nerve damage due to accidents or neurogenic conditions may be alleviated by stimulating the nerves emanating from the patient's spine with neuromodulation. In this example, the electrode is a surgical implant that couples to a nerve bundle extending from the spine. A transdermal patch as described above is used to provide power to the implanted electrode via a transdermal coupling, and may incorporate an on/off switch and a short term battery pack. When the battery of the patch is spent, the entire patch may be replaced, or the battery only may be replaced or recharged.

Nerve regeneration may be induced using the system of the present invention as well. In general, developing nerve growth requires establishing a nerve conduit for growing nerve bundles on the order of several centimeters in length. Nerve growth is stimulated by coupling an electrode to a severed nerve where the electrode is shaped to direct the growth along a desired path. For example, the electrode may form a conduit into which the nerve bundle can grow. The conduit, which is surgically placed in the body adjacent to the severed nerve end, applies current to the nerve end to promote nerve growth over several months.

In addition to stimulating the growth of damaged nerves, neurostimulation according to the present invention may be used to interfere with nerve signals. For example, signals indicating pain may be disrupted, thus reducing discomfort felt by the patient due to disease, injury or surgical procedures. As would be understood by those skilled in the art, to interfere with nerve signals, an electrode is implanted near a desired nerve, and appropriately modulated signals are provided thereto to impair the nerve's ability to carry sensations (e.g., pain) therealong. A biochemical sensor may be also implanted, in a manner similar to the examples discussed in regard to FIGS. 5, 7 and 9, to control actuation of the nerve stimulation to disrupt pain signals. For example, the biochemical sensor may be tuned to sense production of endorphins in the patient's body, and may be designed to interfere with transmission along selected nerves associated with the pain signals stimulating the production of the endorphins. As would be further understood by those skilled in the art, endorphins are produced as a result of pain and their presence is a good indicator that the neuromodulation system should start operating to relieve the patient's discomfort. However, other physiological symptoms of pain may also be sensed to control nerve stimulation to interfere the transmission of pain signals.

In a different embodiment according to the present invention, electrodes may be placed near a nerve or bundle of nerves to either cause or prevent muscle contractions. Artificially stimulated contractions may be useful to exercise muscles that have weakened or lost tone and need to be strengthened to restore various functions. For example, neuromodulation may be used to exercise the pelvic floor muscles in the treatment of hypermobility and other forms of incontinence. To control the function of a target muscle or muscles, electrodes may be coupled to the nerves controlling the target muscles surgically, laparoscopically or percutaneously, depending on the anatomy of the target site, etc. as would be understood by those skilled in the art. An electronic controller may be used to modulate the electric stimuli imparted to the nerves so that the desired muscle reaction is obtained. For example, rhythmic contraction designed to strengthen the target muscles may be induced. An external component including a transdermal coupling as described above may be used to power an implanted component including one or more electrodes, and in some cases to select the modulation and strength of the stimuli as described above.

Certain embodiments according to the present invention thus may be used to treat incontinence through the electrical stimulation of specific targeted muscles. The placement of at least one electrode may be targeted to a specific muscle to obtain the desired automatic exercise regimen. This regimen may be carried out during selected time periods, without patient intervention or disruption of the patient's routines. In the case of treatment for incontinence, the targeted muscles are those of the pelvic floor. For example, the electrode may be placed percutaneously and may be made to travel along the back side of the pubic bone. The electrode may attach to the muscle using various anchoring devices, described below, such that the electrode is placed in contact with the individual targeted muscle.

A different effect may be obtained to prevent muscle spasm, such as those that cause urge incontinence. In this embodiment according to the invention, the electrodes are placed surgically in nerve bundles emanating from the spine or from the associated appendages. In this case, an external electronic module as described above is used to power a implanted component which electrically stimulates one or more nerves associated with the target muscles at a level and a duty cycle appropriate to prevent involuntary contraction of the target muscles to forestall the spasms leading to incontinence. In order to achieve this end, a system as described in regard to any of the embodiments disclosed herein may be employed.

FIG. 10 shows a further exemplary embodiment of an implantable electrode and transdermal receiver according to the present invention. In this example, a nerve couple 910 is placed in contact with a nerve 912. The nerve couple 910 comprises an electrode 922 that carries electrical stimuli to the nerve fibers, and an anchoring element 920 which is deployed after insertion of the electrode 922 in the nerve 912 to retain the nerve couple 910 in place. Alternatively, the electrode 922 may be anchored within muscle fibers so that muscle activity may be induced through direct electrical stimulation. A conductor 908 connects the electrode 922 to an implanted electronics module 906, which is placed just below the surface of the patient's skin 916. The conductor 908 may be surrounded by an insulator 918 to prevent electric energy carried therein from affecting other, non targeted nerves and muscles. It will be apparent to those skilled in the art that a single lead or a dual lead electrode may be used depending on the application, and that a return loop may also be included to provide feedback from the nerve or muscle.

The electronics module 906 may include anchors 914 used to retain the unit in place relative to the surrounding tissues. For example, sutures may be used to keep the module 906 in position under the skin in an area easily accessible by the patient. The patient may then easily place a transdermal patch in the vicinity of the module 906 to transmit power and commands to the implanted module 906. In one exemplary embodiment, the electronics module 906 comprises a transdermal coupling receiver adapted to receive electrical energy from the external transdermal patch. As discussed with reference to the embodiments shown in FIGS. 4-9, the implanted electronics module 906 may also comprise a signal generation module, various sensors, and a radio receiver to obtain control signals transmitted from outside the body. The specific configuration of the implanted electronics module 906 may thus be varied to suit specific requirements of each medical procedure.

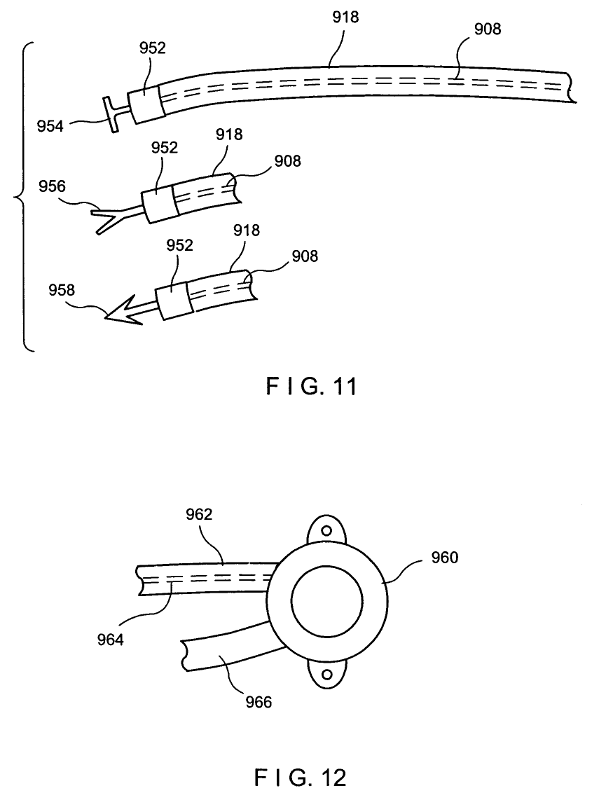

Several variations on the distal end of a conductor 908 with insulator 918 are shown in FIG. 11. Here, the exemplary conductor 908 terminates in an electrode 952 which may comprise a non-insulated wire or a shaped piece of conductive material. An anchoring system adapted to secure the electrode 952 in contact with a targeted muscle is provided, which may comprise, for example, a “T” shaped element 954. Once the “T” shaped anchoring element 954 is deployed in the muscle tissue, it is difficult to remove it, thus a semi permanent anchoring is formed. Prior to deployment, the anchoring element 954 may have a folded configuration 956 which opens to a “T” shape once deployed, or may have a barbed configuration 958 which further enhances the anchoring propertied of the element. In one exemplary embodiment, the anchoring element 954 is made of a non conductive material, to prevent unwanted stimulation.

As described above. the exemplary neuromodulation system according to the invention may be designed to cycle on and off automatically and/or to modulate the electrical stimulus as desired (e.g., according to a particular pattern or algorithm). This stimulus may be used to contract and relax targeted muscles according to a selected exercise regimen to promote muscle strengthening. A muscle strength feedback monitoring system may also be included to determine the effectiveness of the treatment. The system may operate for an extended period of time (e.g., several weeks), until the batteries in the external electronics module are exhausted. The module may then be replaced or recharged. An on/off switch may be provided, so that operation of the neurostimulation system does not interfere with normal muscle contractions (e.g., in the case of incontinence treatment, those associated with normal voiding). Since the internal components may be left in the patient after conclusion of the treatment regimen, a relapse of the medical condition may be treated by simply reactivating the implanted electronics module and nerve couple with a new external patch containing a fresh power supply.

Details of the duty cycle, the modulation and the strength of the electrical stimulus provided by the implanted electronics module may be selected for each specific application. For example, the parameters used to exercise muscles in the treatment of incontinence may be different from those used to block pain signals, etc. As described above, the stimulation parameters may be provided by an electronics module external to the body, or by one implanted together with the implanted transdermal couple.

FIG. 12 shows a different exemplary embodiment of the neurostimulation device according to the invention. This embodiment may be similar to the embodiment described with reference to FIG. 10, however the implanted electronics module 960 of this example uses two separate electrodes and conductors to complete the electric circuit providing the stimuli. The electrode 964 with insulation 962 reached the targeted tissue and forms a first portion of the electric circuit. A return electrode 966 is also provided, which forms a return path for the current directed to the targeted muscle or nerve. Other embodiments described above principally use the patient's body to provide a return path for the current. The present exemplary embodiment may results in a more efficient delivery of the electrical energy, however at the expense of additional complications in implanting the return electrode.

The present invention has been described with reference to specific exemplary embodiments. Those skilled in the art will understand that changes may be made in details, particularly in matters of shape, size, material and arrangement of parts. Accordingly, various modifications and changes may be made to the embodiments. For example, the location of the implanted components of the system may be varied to reflect the location of the nerves and muscles that are to be stimulated. Additional or fewer components may be used, depending on the condition that is being treated by the neurostimulation system. The specifications and drawings are, therefore, to be regarded in an illustrative rather than a restrictive sense.

1. An electrical stimulation system comprising:
an implant adapted for insertion into a body in contact with target tissue, the implant including a transdermal receiver; and
an external driver including a source of stimulation current and a transdermal transmitter transmitted the stimulation current to the transdermal receiver.

2. The electrical stimulation system according to claim 1, further comprising a neural couple of the implant for supplying the stimulation current to a nerve.

3. The electrical stimulation system according to claim 1, wherein the implant includes a muscular stimulation electrode for insertion in muscular tissue.

4. The electrical stimulation system according to claim 1, wherein the source of stimulation current comprises a battery.

5. The electrical stimulation system according to claim 1, wherein the external driver includes a control module controlling the stimulation current.

6. The electrical stimulation system according to claim 5, wherein the control module is adapted to control at least one of an intensity, waveform and duty cycle of the stimulation current.

7. The electrical stimulation system according to claim 1, wherein the implant comprises an electronics module and a lead conductor.

8. The electrical stimulation system according to claim 7, wherein the lead conductor is an elongated conductor comprising an insulated portion and a conductive tip for contacting the target tissue.

9. The electrical stimulation system according to claim 7, wherein the lead conductor comprises an anchoring element to retain the conductor in the target tissue.

10. The electrical stimulation system according to claim 9, wherein the anchoring element is selectively deployable from the lead conductor.

11. The electrical stimulation system according to claim 7, wherein the implant further comprises a return conductor for receiving feedback from the target tissue.

12. The electrical stimulation system according to claim 1, wherein the external driver portion is a flexible planar element adapted to conform to a shape of a portion of skin to which it is attached.

13. The electrical stimulation system according to claim 1, wherein the external driver transmits control signals to the implant via the transdermal transmitter and the transdermal receiver.

14. The electrical stimulation system according to claim 1, wherein the transdermal transmitter and the transdermal receiver include one of a radio frequency transreceiver, a magnetic transreceiver and an induction transreceiver.

15. The electrical stimulation system according to claim 1, wherein the implant includes a biochemical sensor.

16. The electrical stimulation system according to claim 16, wherein the implant includes an electronic controller controlling an applied current applied by the implant based on signals from the biochemical sensor.

17. The electrical stimulation system according to claim 1, wherein the external driver comprises an on/off switch.

18. The electrical stimulation system according to claim 1, wherein the implant includes a telemetry transmitter and the external driver includes a telemetry receiver, the telemetry transmitter transmitting signals to the telemetry receiver.

19. The electrical stimulation system according to claim 18, wherein the signals comprise at least one of neurofeedback signals from a neural couple and signals from a biochemical sensor.

20. An incontinence treatment device, comprising:
an implant including an electrode adapted to be surgically placed adjacent to a target tissue to be stimulated for delivering a stimulation current to the target tissue; and
an external driver transdermally powering and controlling the stimulation current applied by the electrode, the external driver including a power source.

21. The incontinence treatment device according to claim 20, wherein the target tissue is a nerve.

22. The incontinence treatment device according to claim 20, wherein the external driver includes a transmitter transmitting power and control signals to the implant.

23. The incontinence treatment device according to claim 22, wherein the implant includes a receiver and wherein the transmitter provides one of electric, magnetic and inductive energy to the receiver.

24. The incontinence treatment device according to claim 22, wherein the implant includes a receiver and wherein the transmitter provides signals to the receiver to control at least one of a voltage, a current modulation, a duration and a duty cycle of the stimulation current.

25. The incontinence treatment device according to claim 20, wherein the electrode selectively electrically stimulates muscles to control urinary incontinence.

26. The incontinence treatment device according to claim 20, wherein the electrode comprises an anchor.

27. The incontinence treatment device according to claim 20, wherein the electrode comprises a neural couple.

28. The incontinence treatment device according to claim 20, wherein the power source includes a battery.

29. The incontinence treatment device according to claim 22, wherein the implant includes a feedback sensor gathering a feedback signal and a feedback transmitter and wherein the external driver includes a feedback receiver receiving feedback signals from the implant via the feedback transmitter.

30. The incontinence treatment device according to claim 29, wherein the feedback sensor is one of a neuro sensor and a biochemical sensor.

31. The incontinence treatment device according to claim 20, further comprising a proximity sensor to determine an appropriate relative position of the external driver and the implant.

32. A method of electrically stimulating target tissue, comprising:
surgically implanting an implant with an electrode of the implant adjacent to the target tissue, the implant including a receiver;
placing an external driver including a transmitter on a target portion of skin in proximity to the receiver;
providing power to the implant portion transdermally from the external driver via the transmitter and the receiver; and
applying a stimulation current to the target tissue by supplying power received from the external driver to the electrode.

33. The method according to claim 32, further comprising providing a control signal from the external driver to the implant portion, the control signal controlling at least one of a current/voltage modulation, an intensity, a duration and a duty cycle of the stimulation current.

34. The method according to claim 33, further comprising providing a feedback signal from the implant to the external driver.

35. The method according to claim 34, further comprising modifying the control signal in response to the feedback signal.

36. The method according to claim 38, wherein the external driver includes an on/off control allowing a patient to control the application of the stimulation current.
